(19) United States
(12) Patent Application Publication
(10) Pub. No.: US 2010/0210915 A1
(43) Pub. Date: Aug. 19, 2010

(54) SURGICAL LIFT DEVICE TO ASSIST IN SURGICAL ACCESS THROUGH SKIN, TISSUE AND ORGANS

(75) Inventors: Darwin Caldwell, Serra Ricco' (IT); Brain Davies, London (GB)

Correspondence Address: MERCHANT & GOULD PC, P.O. BOX 2903, MINNEAPOLIS, MN, 55402-0903, US

(73) Assignee: Fondazione Istituto Italiano Di Tecnologia, Genova (IT)

(21) Appl. No.: 12/704,490

(22) Filed: Feb. 11, 2010

(30) Foreign Application Priority Data
Feb. 13, 2009 (EP) 09425051.1

(51) Int. Cl. A61B 1/32

(52) U.S. Cl. 600/210

(57) ABSTRACT

A surgical lift device includes a suction member for detachably contacting an external skin surface of a human body wall and holding the external skin surface by application of negative pressure between the suction member and the external skin surface. The suction member has a gripping force sufficient to permit lifting of the human body wall to an elevated position and to hold the human body wall in the elevated position. The suction member has a load-bearing domed structure which determines the lifting of the human body wall during application of negative pressure. The surgical lift device further includes a low friction entry port device arranged on the dome structure, wherein the low friction entry port device has a low friction port fixture for inserting a tool.

This application claims benefit of Ser. No. 09425051.1, filed 13 Feb. 2009 in the European Patent Office and which application is incorporated herein by reference. To the extent appropriate, a claim of priority is made to the above disclosed application.

### FIELD OF THE INVENTION

The present invention relates to a surgical lift device, comprising a suction member for detachably contacting an external skin surface of a human body wall and holding said external skin surface by means of application of negative pressure between said suction member and said external skin surface, said suction member having a gripping force sufficient to permit lifting of said human body wall to an elevated position and to hold said human body wall in said elevated position.

### BACKGROUND OF THE INVENTION

In order to assist in access to organs through the skin, for procedures such as minimally invasive surgery, a process of insufflation is often used in which a pressurised gas is passed through the skin and attached fat layers, in order to allow tools such as those in laparoscopic surgery, to have better access to organs and tissue inside the domed region that is then formed. This procedure is often performed to access the inside of the abdomen, but may also be undertaken to access any other organs, such as lungs or heart. The gas is normally inserted through a trocar which must be sealed in order to prevent the loss of gas. If this loss should happen during surgery, the procedure has to be abandoned or delayed until the pressure is restored.

A further disadvantage of insufflation is that the pressurised gas can be absorbed, causing the patient pain and bloating for some time subsequently. A further disadvantage of insufflation is that the instruments used in surgery must pass through the sealed trocar, leading to a considerable increase in friction between the trocar and the tool for in/out and axial rotation motions, so that much of the sense of touch of the tool against internal tissue is lost. Also, since the skin is tightened like the surface of a drum, any attempt to pivot the tools in pitch and yaw relative to the skin will meet considerable resistance, further degrading any sense of “feel” of tools against internal tissue. As a consequence, the surgeon has to rely primarily on vision to judge any tool/tissue contact and observe any resulting tissue compression in order to judge the magnitude of tool contact forces.

An alternative to insufflation has also been used in which hooks are placed through the skin and attached to cords and levers to lift the skin and fatty tissue away from underlying organs. However, this has not been popular due to the trauma caused to the skin and also the need to provide the hooks with an overhead support which can impede the surgical access.

EP 0 672 385 discloses a surgical lift device of the type defined at the beginning of the description. This device utilizes a suction member for gripping the external skin surface of a human body wall. This removes the need for the pressurised gas inside the human body, avoiding the pain caused by the insufflation process. However, even this device suffers from some drawbacks. In particular, it includes a lifting member for lifting the suction member, which may interfere with other devices or operators' movements during surgical interventions.

### SUMMARY OF THE INVENTION

According to the present invention, it is provided a surgical lifting device of the type defined at the beginning, wherein the suction member has a load-bearing domed structure which determines the lifting of the human body wall during application of negative pressure, and wherein it is further comprised a low friction entry port device arranged on the domed structure, said low friction entry port device comprising a low friction port fixture for inserting a tool. Due to the fact that the domed structure determines the lifting of the human body wall, the surgical lift device does not need any separate lifting member. Therefore, any interference situation during surgical interventions is avoided. Furthermore, the low friction port device allows tools to easily pass through and allows the contact forces to be judged more readily, either through direct contact of tool on tissue or with the aid of additional force sensing enhancement.

Preferably, the domed structure is made of a material which is transparent to visible light.

In accordance with a preferred embodiment, the suction member comprises a flexible porous membrane which extends over the entire base side of the suction member.

According to a further embodiment, the suction member comprises an inner rigid porous membrane.

According to a further preferred embodiment, at least one, and preferably a plurality of apertures are formed through the suction member in order to give access to said external skin surface, said apertures being formed in such a way as to ensure the external skin surface may be sealed to the suction member in the nearby regions around said apertures.

According to an embodiment, the suction member is provided with at least one, and preferably a plurality of flexible tubular walls which respectively surround said apertures and extend from the wall of the domed structure to the base side thereof, said tubular walls being non-permeable to air and having respective terminal portions formed in such a way as to be sealed to said external skin surface. Preferably, the tubular walls are preformed so as to fold like a accordion during application of negative pressure.

According to an alternative embodiment, the suction member is provided with a non-permeable sheet which covers the inner side of the domed structure so that the apertures are covered, said non-permeable sheet being perforable in use for permitting tool insertion.

In accordance with a further embodiment, the low friction entry port device is integrally formed in the dome structure at one of the apertures thereof

In accordance to an alternative embodiment the low friction entry port device comprises a support plate to be removably placed over one of the apertures of said suction member in such a way as to rest on the edge of such aperture, and the low friction port fixture is provided on said support plate.

### BRIEF DESCRIPTION OF THE DRAWINGS

Some preferred, but non-limiting, embodiments of the invention will now be described, with reference to the attached drawings, in which:

FIG. 1 is a plan view sketch of a surgical lift device according to the invention;

FIG. 2 is a sectional view of the device of FIG. 1 in a rest position, taken along the line II-II;

FIG. 3 is a sectional view sketch of the device of FIG. 1 in an operating position, taken along the line II-II;

FIG. 4 in an enlarged view of an aperture of the device of FIG. 1, equipped with a laparoscopic tool;

FIG. 5 is a sectional view sketch of the device of FIG. 1 applied over breast; and

FIG. 6 is a sectional view of another embodiment of a surgical lift device according to the invention.

### DETAILED DESCRIPTION OF PREFERRED EMBODIMENTS

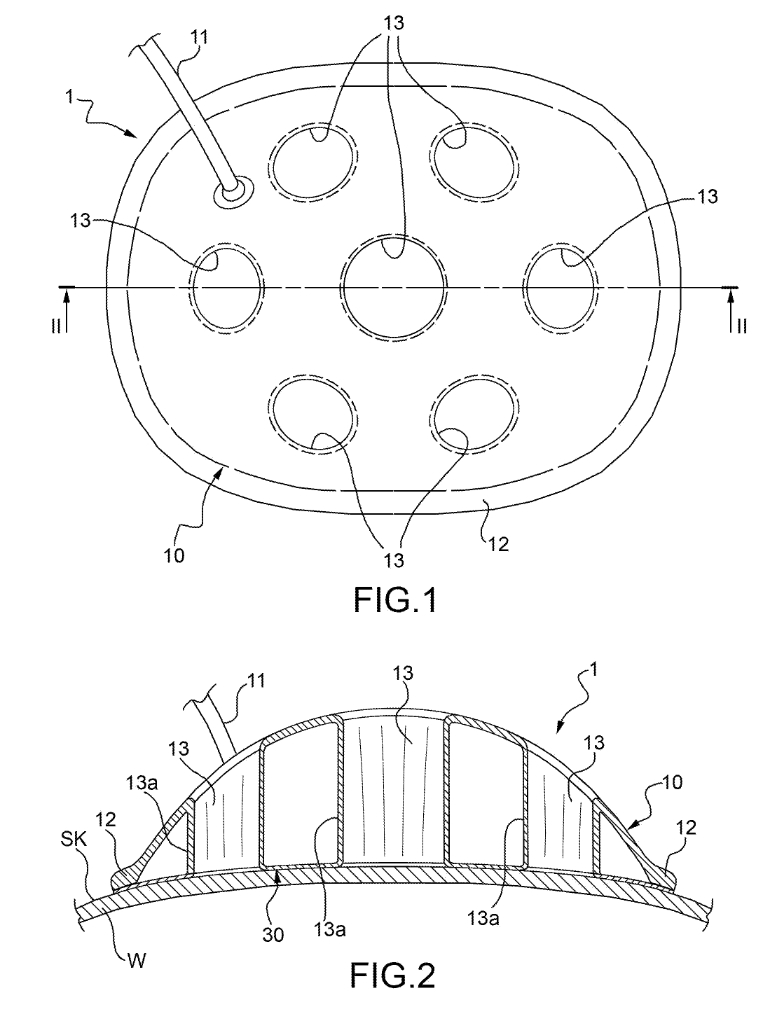

With reference to FIGS. 1 to 5, there is shown a surgical lift device according to the invention, generally indicated with 1.

Surgical lift device 1 comprises a suction member 10 having a domed structure suitable for being placed on an external skin surface SK of a human body wall W, for example the skin surface of the abdominal wall. The suction member 10 is designed in such a way as to be sealed to the skin surface SK. Preferably, the material of the domed structure is transparent to visible light.

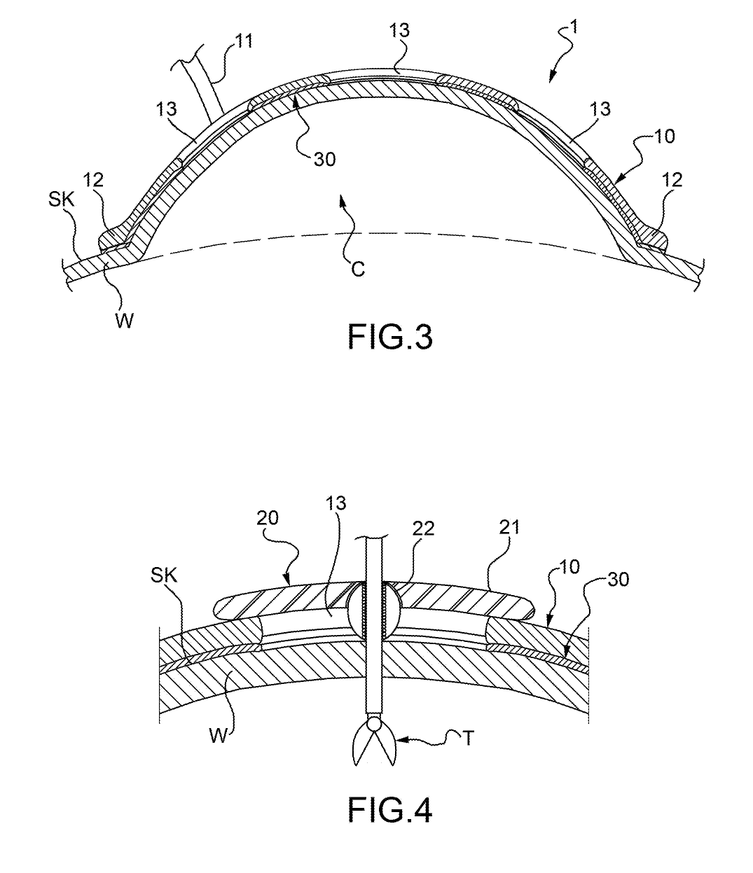

The suction member 10 is connectable to a vacuum source (not shown) through at least one vacuum line 11 for applying a negative pressure to the gap between skin surface SK and the domed structure. As shown in FIG. 3, this negative pressure shall be of a magnitude sufficient to lift the body wall W (i.e. skin together with attached fatty tissue) to conform to the shape of the dome structure leaving a cavity C on the back side of the body wall (the dashed line in FIG. 3 schematically indicates the border of the area occupied by the internal organs). This removes the need for the pressurised gas inside the abdomen, avoiding the pain previously caused.

The domed structure of the suction member 10 is load-bearing (in other words, rigid or semi-rigid), i.e. it does not collapse under the action of the negative pressure, or it collapses to a limited extent with respect to the lifting movement of the human body wall. Therefore, the domed structure determines the lifting of said human body wall during application of negative pressure. This feature removes the need for a separate lifting member, such as that provided in EP 0 672 385.

The domed structure can be provided in a range of shapes and sizes to suit different sized patients or different areas of the body, such as the breast. The edges 12 of the domed structure can be flexible to enhance sealing of the region of negative pressure to the skin surface SK.

At least one, and preferably a plurality of apertures 13 are formed through the suction member 10 in order to give access to the skin surface SK. These apertures 13 are formed in such a way as to ensure the skin surface SK is sealed to the suction member 10 in the nearby regions around the apertures 13. As shown in FIG. 2, this may be obtained by forming flexible tubular walls 13a which extend from the wall of the domed structure to the base side thereof These tubular walls 13a are non-permeable to air. At the base side of the domed structure, the terminal portions of these flexible tubular walls are formed in such a way as to be sealed to the external skin surface SK. The tubular walls 13a are formed so as that they are less likely to cover the apertures 13 as they collapse upon vacuum application. For example, the tubular walls 13a may be preformed so as to fold like a accordian.

The areas of the apertures 13 can be used to insert endoscopes and tools without restriction from skin tension. If a trocar were used in these areas, it would not need to be gas sealed and so simple low-friction features could be used in the trocar to allow tools to pass through and allow the contact forces to be judged more readily, either through direct contact of tool on tissue or with the aid of additional force sensing enhancement. Tool and endoscope in/out and axial rotation motions may be further enhanced by the use of slippery coatings or low friction devices such as recirculating ball-races or screws. Alternatively, if a trocar were not used, a specially designed access port can allow low-force contact with tissue. An additional benefit from the device is that it can facilitate low friction pitch and yaw motions of the laparoscopic tools and endoscopes, without the restriction previously caused by insufflation and stretched skin in the region. This can be enhanced by utilising low friction pivots at the access port or trocar device, allowing an enhanced sense of feel when pivoting the tools in order to contact tissue. A particular embodiment of the invention is shown in FIG. 4. This embodiment uses a region of the domed structure of the suction member 10 as a support for a low friction entry port device 20. This low friction entry port device 20 comprises a support plate 21 to be placed over one of the apertures 13 of the suction member 10 in such a way as to rest on the edge of such aperture. A low friction port fixture 22, such as a low friction pivot, is provided on the support plate 21 for inserting a tool T. In this way, the tool T may be operated through port fixture 22 and aperture 13. According to a further embodiment (not shown), the low friction entry port device may be integrated in the domed structure at one of the apertures 13 thereof It is to be understood that the port device shown in FIG. 4 is only an example; many other kinds of devices are available which may be coupled to the domed structure of the present invention. In any case the tools may either be moved manually or by actuators and control systems that form part of a robotic manipulator.

As shown in the drawings, the suction member 10 may be provided with a flexible porous membrane 30 which extends over the entire base side of the suction member 10. In this case, the negative pressure may be applied between the membrane 30 and the domed structure to facilitate sealing whilst minimising skin trauma. Alternatively or in combination, a rigid porous membrane (not shown) may be used inside the domed structure. In this case the negative pressure may be placed between the domed structure and the rigid membrane. According to a further alternative (not shown), the domed structure is directly placed to the skin, without intermediate membrane. In this case, the negative pressure is directly applied in the gap between the domed structure and the skin surface. Both domed structure and membrane may be transparent to enhance vision of the surgical site. The negative pressure is determined so as to be sufficient to lift the skin to the dome, whilst not being too great to cause the capillaries to burst.

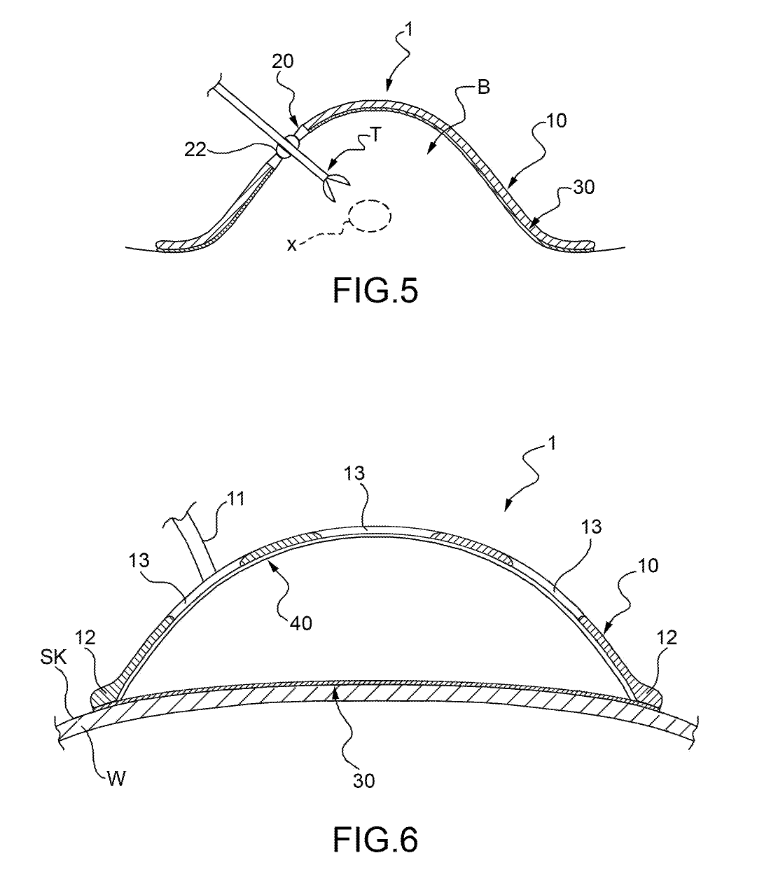

A further benefit of the domed negative pressure structure is that it can be used to condition the tissue or organ to form a constant shape defined by the dome without changing shape, such as due to gravitational effect during a change of pose. An example of this is in the breast diagnostic and surgery, as shown in FIG. 5. In this Figure, B indicates the breast zone, while X indicates the position of a tumour. It is known that in breast diagnostic images may be taken prone whilst a surgical operation may be performed supine. This can lead to a considerable difference in shape of the organ due to gravitational effects when changing pose. The use of the proposed device to condition the breast to a constant form can ensure a consistent shape between imaging and intervention, irrespective of pose.

FIG. 6 shows another embodiment of a surgical lift device according to the invention. Elements corresponding to those of FIGS. 1 to 5 are identified by like reference numerals. The embodiment of FIG. 6 represents an alternative viable and cost-effective means of avoiding air being sucked through apertures 13 when applying negative pressure. This embodiment does not have the tubular walls 13a, but is provided with a thin transparent plastic non-permeable sheet 40 which covers the inner side of the domed structure so that the apertures 13 are covered. Once a position corresponding to that of FIG. 3 is attained, and the porous membrane 30 covers the inner side of the domed structure sealing around the edge of the apertures 13, the sheet 40 can be perforated at 13, permitting tools and telescopes to be passed through the holes and through the skin and tissue.

It is to be understood that the embodiments shown in the Figures are only examples. There are a number of other possible means of sealing the apertures 13 which are available to the person skilled in the art. Some examples comprise sliding covers, a separate external dome to seal the domed structure and apertures until position in FIG. 3 is achieved, partially excised discs, and so on. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A surgical lift device, comprising a suction member for detachably contacting an external skin surface of a human body wall and holding said external skin surface by application of negative pressure between said suction member and said external skin surface, said suction member having a gripping force sufficient to permit lifting of said human body wall to an elevated position and to hold said human body wall in said elevated position, wherein said suction member has a load-bearing domed structure determining the lifting of said human body wall during application of negative pressure, and wherein said surgical lift device further comprises a low friction entry port device arranged on the domed structure, said low friction entry port device including a low friction port fixture for inserting a tool.

2. A surgical lift device according to claim 1, wherein said domed structure is made of a material which is transparent to visible light.

3. A surgical lift device according to claim 1, wherein said suction member comprises a flexible porous membrane extending over an entire base side of the suction member.

4. A surgical lift device according to claim 1, wherein said suction member comprises an inner rigid porous membrane.

5. A surgical lift device according to claim 1, wherein a plurality of apertures are formed through the suction member in order to give access to said external skin surface, said apertures being formed to ensure the external skin surface may be sealed to the suction member in nearby regions around said apertures.

6. A surgical lift device according to claim 5, wherein said suction member is provided with a plurality of flexible tubular walls which respectively surround said apertures and extend from the wall of the domed structure to a base side of the domed structure, said tubular walls being non-permeable to air and having respective terminal portions formed to be sealed to said external skin surface.

7. A surgical lift device according to claim 6, wherein said tubular walls are preformed to fold like an accordian during application of negative pressure.

8. A surgical lift device according to claim 5, wherein said suction member is provided with a non-permeable sheet covering the inner side of the domed structure so that the apertures are covered, said non-permeable sheet being perforable in use for permitting tool insertion.

9. A surgical lift device according to claims 5, wherein said low friction entry port device is integrally formed in the dome structure at one of the apertures.

10. A surgical lift device according to claim 5, wherein said low friction entry port device comprises a support plate removably placed over one of the apertures of said suction member to rest on the edge of such aperture, and said low friction port fixture is provided on said support plate.

11. A surgical lift device according to claim 1, wherein at least one aperture is formed through the suction member in order to give access to said external skin surface, said aperture being formed to ensure the external skin surface may be sealed to the suction member in nearby regions around said aperture.

12. A surgical lift device according to claim 11, wherein said suction member is provided with at least one tubular wall which respectively surrounds said aperture and extends from the wall of the domed structure to a base side of the domed structure, said tubular wall being non-permeable to air and having respective terminal portions formed to be sealed to said external skin surface.
